(19) United States
(12) Patent Application Publication
(10) Pub. No.: US 2010/0055747 A1
(43) Pub. Date: Mar. 4, 2010

(54) Method for Improving Yield of Cellulose Conversion Processes

(76) Inventors: Bradley Kelemen, Palo Alto, CA (US); Edmund A. Larenas, Moss Beach, CA (US); Colin Mitchinson, Palo Alto, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/514,375

(22) PCT Filed: Nov. 13, 2007
(86) PCT No.: PCT/US07/23732

§ 371 Date: May 11, 2009

Related U.S. Application Data
(60) Provisional application No. 60/859,579, filed on Nov. 15, 2006.

(51) Int. Cl. C12P 19/02

(52) U.S. Cl. 435/105

(57) ABSTRACT

The present teachings provide methods of converting cellulosic materials to soluble sugars. Methods for increasing the yield of glucose from the enzymatic saccharification of cellulosic materials is also provided. The present teachings further provide methods of increasing the yield of cellobiose from the enzymatic saccharification of cellulosic materials.

### CROSS-REFERENCE TO RELATED APPLICATIONS

The present application claims benefit of and priority to U.S. Provisional Application Ser. No. 60/858,579, entitled “Method for Improving Yield of Cellulose Conversion Process”, filed Nov. 13, 2006, incorporated herein by reference in its entirety.

### STATEMENT AS TO RIGHTS TO INVENTIONS MADE UNDER FEDERALLY SPONSORED RESEARCH AND DEVELOPMENT

Portions of this work were funded by Subcontract No. ZCO-0-30017-01 with the National Renewable Energy Laboratory under Prime Contract No. DE-AC36-99GO10337 with the U.S. Department of Energy. Accordingly, the United States Government may have certain rights in this invention.

### FIELD

The present teaching relates to methods for improving the yield of desirable sugars in the enzymatic conversion of cellulosic materials.

### BACKGROUND

The production of sugars from cellulosic materials has been known for some time, as has the subsequent fermentation and distillation of these sugars into ethanol. Much of the prior development occurred around the time of World War II when fuels were at a premium in such countries as Germany, Japan and the Soviet Union. These early processes were primarily directed to acid hydrolysis but were fairly complex in their engineering and design and were very sensitive to small variations in process variables, such as temperature, pressure and acid concentrations. A comprehensive discussion of these early processes is presented in “Production of Sugars From Wood Using High-pressure Hydrogen Chloride”, Biotechnology and Bioengineering, Volume XXV, at 2757-2773 (1983).

The abundant supply of petroleum in the period from World War II through the early 1970s slowed ethanol conversion research. However, due to the oil crisis of 1973, researchers increased their efforts to develop processes for the utilization of wood and agricultural byproducts for the production of ethanol as an alternate energy source. This research was especially important for development of ethanol as a gasoline additive to reduce the dependency of the United States upon foreign oil production, to increase the octane rating of fuels, and to reduce exhaust pollutants as an environmental measure.

Concurrently with the “oil crisis,” as it became known, the Environmental Protection Agency of the United States promulgated regulations requiring the reduction of lead additives in an effort to reduce air pollution. Insofar as ethanol is virtually a replacement of lead, some refineries have selected ethanol as the substitute, especially since it can easily be introduced into a refinery's operation without costly capital equipment investment.

In addition to improving the high pressure and high temperature gas saccharification processes developed decades ago, current research is directed primarily at enzymatic conversion processes. These processes employ enzymes from a variety of organisms, such as mesophilic and thermophilic fungi, yeast and bacteria, which degrade the cellulose into fermentable sugars. Uncertainty still remains with these processes and their ability to be scaled up for commercialization as well as their inefficient rates of ethanol production.

Cellulose and hemicellulose are the most abundant plant materials produced by photosynthesis. They can be degraded for use as an energy source by numerous microorganisms, including bacteria, yeast and fungi, which produce extracellular enzymes capable of hydrolysis of the polymeric substrates to monomeric sugars (Aro et al., 2001). Organisms are often restrictive with regard to which sugars they use and this dictates which sugars are best to produce during conversion. As the limits of non-renewable resources approach, the potential of cellulose to become a major renewable energy resource is enormous (Krishna et al., 2001). The effective utilization of cellulose through biological processes is one approach to overcoming the shortage of foods, feeds, and fuels (Ohmiya et al., 1997).

Cellulases are enzymes that hydrolyze cellulose (beta-1,4-glucan or beta D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and beta-glucosidases ([beta]-D-glucoside glucohydrolase; EC 3.2.1.21) (“BG”). (Knowles et al., 1987 and Shulein, 1988). Endoglucanases act mainly on the amorphous parts of the cellulose fiber, whereas cellobiohydrolases are also able to degrade crystalline cellulose.

Cellulases have also been shown to be useful in degradation of cellulose biomass to ethanol (wherein the cellulases degrade cellulose to glucose and yeast or other microbes further ferment the glucose into ethanol), in the treatment of mechanical pulp (Pere et al., 1996), for use as a feed additive (WO 91/04673) and in grain wet milling. Separate saccharification and fermentation is a process whereby cellulose present in biomass, e.g., corn stover, is converted to glucose and subsequently yeast strains convert glucose into ethanol. Simultaneous saccharification and fermentation is a process whereby cellulose present in biomass, e.g., corn stover, is converted to glucose and, at the same time and in the same reactor, yeast strains convert glucose into ethanol. Ethanol production from readily available sources of cellulose provides a stable, renewable fuel source.

Cellulases are known to be produced by a large number of bacteria, yeast and fungi. Certain fungi produce a complete cellulase system (i.e., a whole cellulase) capable of degrading crystalline forms of cellulose. In order to efficiently convert crystalline cellulose to glucose the complete cellulase system comprising components from each of the CBH, EG and BG classifications is required, with isolated components less effective in hydrolyzing crystalline cellulose (Filho et al., 1996). In particular, the combination of EG-type cellulases and CBH-type cellulases interact to more efficiently degrade cellulose than either enzyme used alone (Wood, 1985; Baker et al., 1994; and Nieves et al., 1995).

Additionally, cellulases are known in the art to be useful in the treatment of textiles for the purposes of enhancing the cleaning ability of detergent compositions, for use as a softening agent, for improving the feel and appearance of cotton fabrics, and the like (Kumar et al., 1997). Cellulase-containing detergent compositions with improved cleaning performance (U.S. Pat. No. 4,435,307; GB App. Nos. 2,095,275 and 2,094,826) and for use in the treatment of fabric to improve the feel and appearance of the textile (U.S. Pat. Nos. 5,648,263, 5,691,178, and 5,776,757, and GB App. No. 1,358,599), have been described.

Hence, cellulases produced in fungi and bacteria have received significant attention. In particular, fermentation of Trichoderma spp. (e.g., Trichoderma longibrachiatum or Trichoderma reesei) has been shown to produce a complete cellulase system capable of degrading crystalline forms of cellulose. Over the years, Trichoderma cellulase production has been improved by classical mutagenesis, screening, selection and development of highly refined, large scale inexpensive fermentation conditions. While the multi-component cellulase system of Trichoderma spp. is able to hydrolyze cellulose to glucose, there are cellulases from other microorganisms, particularly bacterial strains, with different properties for efficient cellulose hydrolysis, and it would be advantageous to express these proteins in a filamentous fungus for industrial scale cellulase production. However, the results of many studies demonstrate that the yield of bacterial enzymes from filamentous fungi is low (Jeeves et al., 1991).

Soluble sugars, such as glucose and cellobiose, have a multitude of uses in industry for the production of chemicals and biological products. The optimization of cellulose hydrolysis allows for the use of lower quantities of enzyme and improved cost effectiveness for the production of soluble sugars. Despite the development of numerous approaches, there remains a need in the art for improving the yield of soluble sugars obtained from cellulosic materials.

### SUMMARY

The present teachings provide methods for increasing the yield of soluble sugars from the enzymatic saccharification of cellulosic starting materials by incubating a cellulosic substrate or a pretreated cellulosic substrate with a cellulase at a temperature at or about the thermal denaturation temperature of the cellulase. The present teachings also provide methods for increasing the yield of glucose from the enzymatic saccharification of cellulosic starting materials by incubating a cellulosic substrate or a pretreated cellulosic substrate with a cellulase at a temperature at or about the thermal denaturation temperature of the cellulase.

Also provided are methods for converting a cellulosic material to glucose by combining a cellulosic material with a cellulase incubating the cellulosic material and cellulase combination at a temperature greater than about 38° C. to cause a hydrolysis reaction to convert at least 20% of said cellulosic material to soluble sugars, wherein the fraction of glucose is at least 0.75 relative to the soluble sugars. The present teaching further provide methods for converting a cellulosic material to cellobiose by combining a cellulosic material with enzyme mixture comprising an endoglucanase 1, incubating the cellulosic material and cellulase combination cause a hydrolysis reaction to convert up to 50% of the cellulosic material to soluble sugars, wherein fraction of glucose is less than about 0.5 relative to said soluble sugars.

The cellulases can be whole cellulases, cellulase mixtures, or combinations thereof produced by microorganisms from the genii Aspergillus, Trichoderma, Fusarium, Chrysosporium, Penicillium, Humicola, Neurospora, or alternative sexual forms thereof such as Emericella and Hypocrea (See, Kuhls et al., 1996). Preferably, species such as Acidothermus cellulolyticus, Thermobifida fusca, Humicola grisea or Trichoderma reesei may be used.

These and other features of the present teachings are provided herein.

### BRIEF DESCRIPTION OF THE DRAWINGS

The skilled artisan will understand that the drawings are for illustration purposes only and are not intended to limit the scope of the present teachings in anyway.

FIGS. 1A-B show the conversion of dilute acid treated corn stover to soluble sugars by 3.3 mg/g of whole cellulase from Trichoderma reesei over-expressing beta-glucosidase, at 38° C. (open symbols) and 53° C. (closed symbols).

FIGS. 2A-B show the conversion of dilute acid treated corn stover to soluble sugars by 12 mg/g of whole cellulase from Trichoderma reesei over-expressing beta-glucosidase, at 38° C. (open symbols) and 53° C. (closed symbols).

FIGS. 3A-B show the conversion of dilute acid treated corn stover to soluble sugars by 18 mg/g of whole cellulase from Trichoderma reesei over-expressing beta-glucosidase 1, 38° C. (open symbols) and 53° C. (closed symbols).

FIGS. 4A-B show the conversion of dilute acid treated corn stover to soluble sugars by 20 mg/g of whole cellulase from Trichoderma reesei over-expressing beta-glucosidase, at 38° C. (open symbols) and 53° C. (closed symbols).

FIGS. 5A-B show the conversion of dilute acid treated corn stover to soluble sugars by 20 mg/g of whole cellulase from Trichoderma reesei over-expressing beta-glucosidase, at 38° C. (open symbols) and 53° C. (closed symbols).

FIGS. 6A-B show the conversion of dilute acid treated corn stover to soluble sugars by 12 mg/g whole cellulase from Trichoderma reesei, at 38° C. (open symbols) and 53° C. (closed symbols).

FIGS. 7A-B show the conversion of dilute acid treated corn stover to soluble sugars by 12 mg/g of whole cellulase from Trichoderma reesei expressing a CBH1-E1 fusion protein, 38° C. (open symbols) and 53° C. (closed symbols).

FIGS. 8A-B show the conversion of dilute acid treated corn stover to soluble sugars by 15 mg/g of an enzyme mixture of either EG1 and T. reesei CBH1 (squares) or E1 and H. grisea CBH1 (circles) at 38° C. (open symbols) and 65° C. (closed symbols).

FIGS. 9A-B show the conversion of dilute acid treated corn stover to soluble sugars by 15 mg/g of an enzyme mixture of either EG1, T. reesei CBH1 and T. reesei CBH2 (squares) or E1, H. grisea CBH1 and T. reesei CBH2 (circles) at 38° C. (open symbols) and 65° C. (closed symbols).

FIGS. 10A-B show the conversion of dilute acid treated corn stover to soluble sugars by 15 mg/g of an enzyme mixture of either EG1, T. reesei CBH1 (squares) and T. fusca E3 or E1, H. grisea CBH1 and T. fusca E3 (circles) at 38° C. (open symbols) and 65° C. (closed symbols).

FIGS. 11A-F show the conversion of dilute acid treated corn stover to soluble sugars by a Trichoderma reesei strain at 53° C. (closed symbols) and 59° C. (open symbols)

FIGS. 12A-F. The conversion of dilute acid treated corn stover to soluble sugars by a whole cellulase from Trichoderma reesei expressing a CBH1-E1 fusion protein, at 53° C. (closed symbols) and 59° C. (open symbols).

### DETAILED DESCRIPTION OF VARIOUS EMBODIMENTS

Unless defined otherwise, all technical and scientific terms used herein have the same meaning as commonly understood by one of ordinary skill in the art to which this invention belongs. Singleton, et al., Dictionary of Microbiology and Molecular Biology, 2d Ed., John Wiley and Sons, New York (1994), and Hale & Marham, The Harper Collins Dictionary Of Biology, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole.

The term “cellulase” refers to a category of enzymes capable of hydrolyzing cellulose (beta-1,4-glucan or beta D-glucosidic linkages) polymers to shorter cello-oligosaccharide oligomers, cellobiose and/or glucose.

The term “exo-cellobiohydrolase” (CBH) refers to a group of cellulase enzymes classified as EC 3.2.1.91. These enzymes are also known as exoglucanases or cellobiohydrolases. CBH enzymes hydrolyze cellobiose from the reducing or non-reducing end of cellulose. In general a CBHI type enzyme preferentially hydrolyzes cellobiose from the reducing end of cellulose and a CBHII type enzyme preferentially hydrolyzes the non-reducing end of cellulose.

The term “cellobiohydrolase activity” is defined herein as a 1,4-D-glucan cellobiohydrolase (E.C. 3.2.1.91) activity which catalyzes the hydrolysis of 1,4-beta-D-glucosidic linkages in cellulose, cellotetriose, or any beta-1,4-linked glucose containing polymer, releasing cellobiose from the ends of the chain. For purposes of the present invention, cellobiohydrolase activity can be determined by release of water-soluble reducing sugar from cellulose as measured by the PHBAH method of Lever et al., 1972, Anal. Biochem. 47: 273-279. A distinction between the exoglucanase mode of attack of a cellobiohydrolase and the endoglucanase mode of attack can be made by a similar measurement of reducing sugar release from substituted cellulose such as carboxymethyl cellulose or hydroxyethyl cellulose (Ghose, 1987, Pure & Appl. Chem. 59: 257-268). A true cellobiohydrolase will have a very high ratio of activity on unsubstituted versus substituted cellulose (Bailey et al, 1993, Biotechnol. Appl. Biochem. 17: 65-76).

The term “endoglucanase” (EG) refers to a group of cellulase enzymes classified as EC 3.2.1.4. An EG enzyme hydrolyzes internal beta-1,4 glucosidic bonds of the cellulose. The term “endoglucanase” is defined herein as an endo-1,4-(1,3;1,4)-beta-D-glucan 4-glucanohydrolase (E.C. No. 3.2.1.4) which catalyses endohydrolysis of 1,4-beta-D-glycosidic linkages in cellulose, cellulose derivatives (for example, carboxy methyl cellulose), lichenin, beta-1,4 bonds in mixed beta-1,3 glucans such as cereal beta-D-glucans or xyloglucans, and other plant material containing cellulosic components. For purposes of the present invention, endoglucanase activity can be determined using carboxymethyl cellulose (CMC) hydrolysis according to the procedure of Ghose, 1987, Pure and Appl. Chem. 59: 257-268.

The term “beta-glucosidase” is defined herein as a beta-D-glucoside glucohydrolase (E.C. 3.2.1.21) which catalyzes the hydrolysis of cellobiose with the release of beta-D-glucose. For purposes of the present invention, beta-glucosidase activity may be measured by methods known in the art, e.g., HPLC.

“Cellulolytic activity” encompasses exoglucanase activity, endoglucanase activity or both types of enzyme activity, as well as beta-glucosidase activity.

Many microbes make enzymes that hydrolyze cellulose, including the bacteria Acidothermus, Thermobifida, Bacillus, and Cellulomonas; Streptomyces; yeast such as a Candida, Kluyveromyces, Pichia, Saccharomyces, Schizosaccharomyces, or Yarrowia and the fungi Acremonium, Aspergillus, Aureobasidium, Chrysosporium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma, or alternative sexual forms thereof such as Emericella and Hypocrea (See, Kuhls et al., 1996).

A “non-naturally occurring” composition encompasses those compositions produced by: (1) combining component cellulolytic enzymes either in a naturally occurring ratio or non-naturally occurring, i.e., altered, ratio; or (2) modifying an organism to overexpress or underexpress one or more cellulolytic enzyme; or (3) modifying an organism such that at least one cellulolytic enzyme is deleted or (4) modifying an organism to express a heterologous component cellulolytic enzyme. The component cellulolytic enzymes may be provided as isolated polypeptides prior to combining to form the non-naturally occurring composition.

We have found, in part, that increased saccharification temperature both increases the yield of glucose from cellulosic materials and also results in improved overall conversion of cellulose such that the fraction of glucose in the conversion product is increased at higher incubation temperatures.

The present teachings provide methods for increasing the yield of soluble sugars from the enzymatic saccharification of cellulosic starting materials by incubating a cellulosic substrate or a pretreated cellulosic substrate with a cellulase at a temperature at or about the thermal denaturation temperature of the cellulase. The present teachings further provide methods for increasing the yield of glucose from the enzymatic saccharification of cellulosic starting materials by incubating a cellulosic substrate or a pretreated cellulosic substrate with a cellulase at a temperature at or about the thermal denaturation temperature of the cellulase.

In the methods of the present disclosure, the cellulosic material can be any cellulose containing material. The cellulosic material can include, but is not limited to, cellulose, hemicellulose, and lignocellulosic materials. In some embodiments, the cellulosic materials include, but are not limited to, biomass, herbaceous material, agricultural residues, forestry residues, municipal solid waste, waste paper, and pulp and paper residues. In some embodiments, the cellulosic material includes wood, wood pulp, papermaking sludge, paper pulp waste streams, particle board, corn stover, corn fiber, rice, paper and pulp processing waste, woody or herbaceous plants, fruit pulp, vegetable pulp, pumice, distillers grain, grasses, rice hulls, sugar cane bagasse, cotton, jute, hemp, flax, bamboo, sisal, abaca, straw, corn cobs, distillers grains, leaves, wheat straw, coconut hair, algae, switchgrass, and mixtures thereof (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York).

The cellulosic material can be used as is or may be subjected to pretreatment using methods known in the art. Such pretreatments include chemical, physical, and biological pretreatment. For example, physical pretreatment techniques can include without limitation various types of milling, crushing, steaming/steam explosion, irradiation and hydrothermolysis. Chemical pretreatment techniques can include without limitation dilute acid, alkaline, organic solvent, ammonia, sulfur dioxide, carbon dioxide, and pH-controlled hydrothermolysis. Biological pretreatment techniques can include without limitation applying lignin-solubilizing microorganisms. The pretreatment can occur from several minutes to several hours, such as from about 1 hour to about 120.

In one embodiment, the pretreatment may be by elevated temperature and the addition of either of dilute acid, concentrated acid or dilute alkali solution. The pretreatment solution can added for a time sufficient to at least partially hydrolyze the hemicellulose components and then neutralized

In some embodiments, the pretreatment is selected from a group consisting of steam explosion, pulping, grinding, acid hydrolysis, and combinations thereof.

The cellulase is reacted with the cellulosic material at about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C. In some embodiments the enzymes are reacted with substrate at or about the thermal denaturation temperature of the cellulase. The pH may range from about pH 5, about pH 5.5, about pH 6, about pH 6.5, about pH 7, about pH 7.5, about pH 8.0, to about pH 8.5. Generally, the pH range will be from about pH 4.5 to about pH 9. Incubation of the cellulase under these conditions results in release or liberation of substantial amounts of the soluble sugar from the cellulosic material. By substantial amount is intended at least 20%, 30%, 40%, 50%, 60%, 70%, 80%, 90%, 95% or more of soluble sugar is available sugar.

The cellulase treatment may occur from several minutes to several hours, such as from about 0.1 hour to about 120 hours, preferably about 12 hours to about 72 hours, more preferably about 24 to 48 hours.

The amount of cellulase is a function of the enzyme(s) applied and the reaction time and conditions given. Preferably, the cellulase(s) may be dosed in a total amount of from about 2-40 mg/g cellulosic material.

In the methods of the present disclosure, the cellulase can be whole cellulase, a whole cellulase supplemented with one or more enzyme activities, and cellulase mixtures. In some embodiments, the cellulase can be a whole cellulase preparation. As used herein, the phrase “whole cellulase preparation” refers to both naturally occurring and non-naturally occurring cellulase containing compositions. A “naturally occurring” composition is one produced by a naturally occurring source and which comprises one or more cellobiohydrolase-type, one or more endoglucanase-type, and one or more beta-glucosidase components wherein each of these components is found at the ratio produced by the source. A naturally occurring composition is one that is produced by an organism unmodified with respect to the cellulolytic enzymes such that the ratio of the component enzymes is unaltered from that produced by the native organism.

In general, the cellulases can include, but are not limited to: (i) endoglucanases (EG) or 1,4-β-d-glucan-4-glucanohydrolases (EC 3.2.1.4), (ii) exoglucanases, including 1,4-β-d-glucan glucanohydrolases (also known as cellodextrinases) (EC 3.2.1.74) and 1,4-β-d-glucan cellobiohydrolases (exo-cellobiohydrolases, CBH) (EC 3.2.1.91), and (iii) β-glucosidase (BG) or β-glucoside glucohydrolases (EC 3.2.1.21).

In the present disclosure, the cellulase can be from any microorganism that is useful for the hydrolysis of a cellulosic material. In some embodiments, the cellulase is a filamentous fungi whole cellulase. “Filamentous fungi” include all filamentous forms of the subdivision Eumycota and Oomycota.

In some embodiments, the cellulase is a Acremonium, Aspergillus, Emericella, Fusarium, Humicola, Mucor, Myceliophthora, Neurospora, Scytalidium, Thielavia, Tolypocladium, or Trichoderma species, whole cellulase.

In some embodiments, the cellulase is an Aspergillus aculeatus, Aspergillus awamori, Aspergillus foetidus, Aspergillus japonicus, Aspergillus nidulans, Aspergillus niger, or Aspergillus oryzae whole cellulase. In another aspect, cellulase is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum whole cellulase. In another aspect, the cellulase is a Humicola insolens, Humicola lanuginosa, Mucor miehei, Myceliophthora thermophila, Neurospora crassa, Scytalidium thermophilum, or Thielavia terrestris whole cellulase. In another aspect, the cellulase a Trichoderma harzianum, Trichoderma koningii, Trichoderma longibrachiatum, Trichoderma reesei e.g., RL-P37 (Sheir-Neiss et al., Appl. Microbiol. Biotechnology, 20 (1984) pp. 46-53; Montenecourt B. S., Can., 1-20, 1987), QM9414 (ATCC No. 26921), NRRL 15709, ATCC 13631, 56764, 56466, 56767, or Trichoderma viride e.g., ATCC 32098 and 32086, whole cellulase.

In some embodiments, the cellulase is a Trichoderma reesei RutC30 whole cellulase, which is available from the American Type Culture Collection as Trichoderma reesei ATCC 56765.

In the present disclosure, the cellulase can be from any microorganism cultivation method known in the art resulting in the expression of enzymes capable of hydrolyzing a cellulosic material. Fermentation can include shake flask cultivation, small- or large-scale fermentation, such as continuous, batch, fed-batch, or solid state fermentations in laboratory or industrial fermenters performed in a suitable medium and under conditions allowing the cellulase to be expressed or isolated.

Generally, the microorganism is cultivated in a cell culture medium suitable for production of enzymes capable of hydrolyzing a cellulosic material. The cultivation takes place in a suitable nutrient medium comprising carbon and nitrogen sources and inorganic salts, using procedures known in the art. Suitable culture media, temperature ranges and other conditions suitable for growth and cellulase production are known in the art. As a non-limiting example, the normal temperature range for the production of cellulases by Trichoderma reesei is 24° C. to 28° C.

Certain fungi produce complete cellulase systems which include exo-cellobiohydrolases or CBH-type cellulases, endoglucanases or EG-type cellulases and beta-glucosidases or BG-type cellulases (Schulein, 1988). However, sometimes these systems lack CBH-type cellulases, e.g., bacterial cellulases also typically include little or no CBH-type cellulases. In addition, it has been shown that the EG components and CBH components synergistically interact to more efficiently degrade cellulose. See, e.g., Wood, 1985. The different components, i.e., the various endoglucanases and exocellobiohydrolases in a multi-component or complete cellulase system, generally have different properties, such as isoelectric point, molecular weight, degree of glycosylation, substrate specificity and enzymatic action patterns.

In some embodiments, the cellulase is used as is produced by fermentation with no or minimal recovery and/or purification. For example, once cellulases are secreted by a cell into the cell culture medium, the cell culture medium containing the cellulases can be used. In some embodiments the whole cellulase preparation comprises the unfractionated contents of fermentation material, including cell culture medium, extracellular enzymes and cells. Alternatively, the whole cellulase preparation can be processed by any convenient method, e.g., by precipitation, centrifugation, affinity, filtration or any other method known in the art. In some embodiments, the whole cellulase preparation can be concentrated, for example, and then used without further purification. In some embodiments the whole cellulase preparation comprises chemical agents that decrease cell viability or kills the cells. In some embodiments, the cells are lysed or permeabilized using methods known in the art.

A cellulase containing an enhanced amount of cellobiohydrolase and/or beta-glucosidase finds utility in ethanol production. Ethanol from this process can be further used as an octane enhancer or directly as a fuel in lieu of gasoline which is advantageous because ethanol as a fuel source is more environmentally friendly than petroleum derived products. It is known that the use of ethanol will improve air quality and possibly reduce local ozone levels and smog. Moreover, utilization of ethanol in lieu of gasoline can be of strategic importance in buffering the impact of sudden shifts in non-renewable energy and petrochemical supplies.

Ethanol can be produced via saccharification and fermentation processes from cellulosic biomass such as trees, herbaceous plants, municipal solid waste and agricultural and forestry residues. However, the ratio of individual cellulase enzymes within a naturally occurring cellulase mixture produced by a microbe may not be the most efficient for rapid conversion of cellulose in biomass to glucose. It is known that endoglucanases act to produce new cellulose chain ends which themselves are substrates for the action of cellobiohydrolases and thereby improve the efficiency of hydrolysis by the entire cellulase system. Therefore, the use of increased or optimized cellobiohydrolase activity may greatly enhance the production of ethanol.

Ethanol can be produced by enzymatic degradation of biomass and conversion of the released saccharides to ethanol. This kind of ethanol is often referred to as bioethanol or biofuel. It can be used as a fuel additive or extender in blends of from less than 1% and up to 100% (a fuel substitute).

Enhanced cellulose conversion may be achieved at higher temperatures using the CBH polypeptides described in, for example, any one of the following US Patent Publications US20050054039, US20050037459, US20060205042, US20050048619A1 and US20060218671. Methods of overexpressing beta-glucosidase are known in the art. See, for example, U.S. Pat. No. 6,022,725. See also, for example, US20050214920.

In some embodiments, the cellulase is a exo-cellobiohydrolase fusion protein, suitable examples, included, CBH1 and Acidothermus cellulolyticus endoglucanase or a Thermobifida fusca endoglucanase, CBH1 and Acidothermus cellulolyticus endoglucanase and particularly an Acidothermus cellulolyticus E1 or GH74 endoglucanase (see for example, US Patent Publication No. 20060057672).

In some embodiments, the cellulase mixture comprises a cellulase selected from Trichoderma reesei Endoglucanase 1 (EG1), Trichoderma reesei cellobiohydrolase 1 (CBH1) and Trichoderma reesei cellobiohydrolase 2 (CBH2), Humicola grisea cellobiohydrolase 1 (CBH1) and Acidothermus cellulolyticus endoglucanase E1 (E1), Thermomonospera fusca E3 exocellulase, and combinations thereof.

The methods of the present disclosure can be used in the production of monosaccharides, disaccharides, and polysaccharides as chemical, fermentation feedstocks for microorganism, and inducers for the production of proteins, organic products, chemicals and fuels, plastics, and other products or intermediates. In particular, the value of processing residues (dried distillers grain, spent grains from brewing, sugarcane bagasse, etc.) can be increased by partial or complete solubilization of cellulose or hemicellulose. In addition to ethanol, some chemicals that can be produced from cellulose and hemicellulose include, acetone, acetate, glycine, lysine, organic acids (e.g., lactic acid), 1,3-propanediol, butanediol, glycerol, ethylene glycol, furfural, polyhydroxyalkanoates, cis, cis-muconic acid, animal feed and xylose.

The present teaching further provide methods for converting a cellulosic material to glucose comprising combining a cellulosic material with a cellulase, incubating said cellulosic material and cellulase combination, cause a hydrolysis reaction to convert cellulosic material to soluble sugars, wherein the said soluble sugars comprises glucose and cellobiose and the fraction of glucose is at least 0.75 relative to said soluble sugars.

The present teaching further provide methods for converting a cellulosic material to cellobiose, comprising combining a cellulosic material with a cellulase mixture comprising an endoglucanase 1. In some embodiments, the endoglucanase 1 can comprise an Acidothermus cellulolyticus E1 endoglucanase, including those described in U.S. Pat. No. 5,536,655 and 6,013,860, and Patent Application Publication Nos. 2003/0109011, 2006/0026715, 20060057672.

In some embodiments, the methods of the present disclosure further comprise the step of determining the amount of glucose and or soluble sugars.

Also provided are methods of converting a cellulosic material to glucose comprising the steps of combining a cellulosic material with a cellulase such that the resulting combination of cellulosic material and cellulase has 1% to about 30% cellulose by weight; and incubating said cellulosic material and cellulase combination at a temperature greater than about 38° C. to about 100° C. for about 0.1 hours to about 96 hours at a pH of from about 4 to about 9 to cause a hydrolysis reaction to convert at least 20% of said cellulosic material to soluble sugars, wherein said soluble sugars comprises glucose and cellobiose, and the fraction of glucose is at least 0.75 relative to said soluble sugars.

Provided herein are methods of converting a cellulosic material to cellobiose comprising the steps of combining a cellulosic material with a cellulase mixture comprising an endoglucanase 1 such that the resulting combination of cellulosic material and cellulase mixture has 1% to about 30% cellulose by weight; and incubating said cellulosic material and cellulase combination at a temperature less than about 100° C. to about 25° C. for about 0.1 hours to about 96 hours at a pH of from about 4 to about 9 to cause a hydrolysis reaction to convert up to 50% of said cellulosic material to soluble sugars, wherein said soluble sugars comprises glucose and cellobiose and the fraction of glucose is less than about 0.5 relative to said soluble sugars.

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein.

Aspects of the present teachings may be further understood in light of the following examples, which should not be construed as limiting the scope of the present teachings. It will be apparent to those skilled in the art that many modifications, both to materials and methods, may be practiced without departing from the present teachings.

### EXAMPLES

Cellulose conversion was evaluated by techniques known in the art. See, for example, Baker et al, Appl Biochem Biotechnol 70-72:395-403 (1998) and as described below. One hundred fifty microliters of substrate per well was loaded into a flat-bottom 96-well microtiter plate (MTP) using a repeater pipette. Twenty microliters of appropriately diluted enzyme solution was added on top. The plates were covered with aluminum plate sealers and placed in incubators at either test temperature, with shaking, for the times specified. The reaction was terminated by adding 100 μl 100 mM Glycine pH 10 to each well. With thorough mixing, the contents thereof were filtered through a Millipore 96-well filter plate (0.45 μm, PES). The filtrate was diluted into a plate containing 100 μl 10 mM Glycine pH 10 and the amount of soluble sugars produced measured by HPLC. The Agilent 1100 series HPLCs were all equipped with a de-ashing/guard column (Biorad #125-0118) and an Aminex lead based carbohydrate column (Aminex HPX-87P). The mobile phase was water with a 0.6 ml/min flow rate.

Pretreated corn stover (PCS)—Corn stover was pretreated with 2% w/w H₂SO₄ as described in Schell, D. et al., J. Appl. Biochem. Biotechnol. 105:69-86 (2003) and followed by multiple washes with deionized water to obtain a pH of 4.5. Sodium acetate was added to make a final concentration of 50 mM and the solution was titrated to pH 5.0. The cellulose concentration in the reaction mixture was approximately 7%.

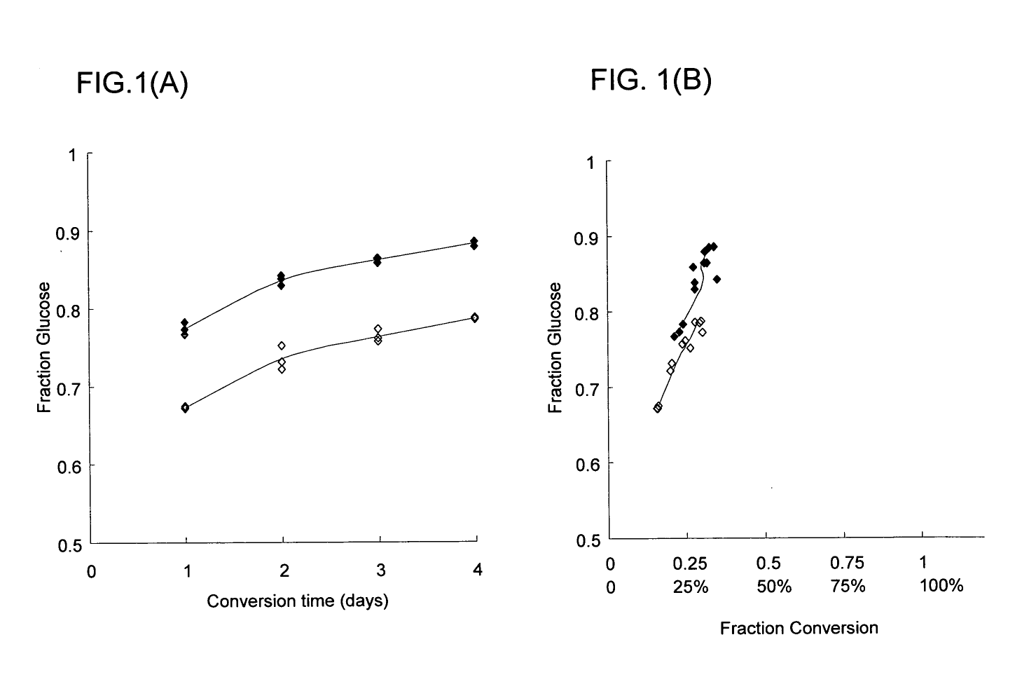

Using the following cellulases: Trichoderma reesei whole cellulase over-expressing beta-glucosidase 1 (WC-BGL1) (see for example, U.S. Pat. No. 6,022,725, Trichoderma reesei whole cellulase expressing a CBH1-E1 fusion protein (WC-CBH1-E1) (see for example, US Patent Publication No. 20060057672), Trichoderma reesei Endoglucanase 1 (EG1), Trichoderma reesei cellobiohydrolase 1 (CBH1) and Trichoderma reesei cellobiohydrolase 2 (CBH2), Humicola grisea cellobiohydrolase 1 (CBH1) and Acidothermus cellulolyticus endoglucanase E1 (E1), Thermomonospera fusca E3 exocellulase. The amount of enzyme was provided in milligrams per gram cellulose. The results of are summarized in FIGS. 1-12. The ordinate represents the fraction of glucose with respect to the total sugar (wt/wt basis). For example, in FIG. 1-10, (A) the ordinate represents the length of conversion time and in FIG. 1-10, (B) the abscissa represents the total soluble sugar conversion that is observed (each incubation time is not explicitly labeled but a later incubation time is indicated by higher conversion).

FIGS. 1A-B show the conversion of dilute acid treated corn stover to soluble sugars by 3.3 mg/g whole cellulase from Trichoderma reesei over-expressing beta-glucosidase, at 38° C. (open symbols) and 53° C. (closed symbols). T. reesei whole cellulase with elevated β-glucosidase levels converts acid-pretreated corn stover to a higher fraction of glucose at 53° C. than at 38° C.

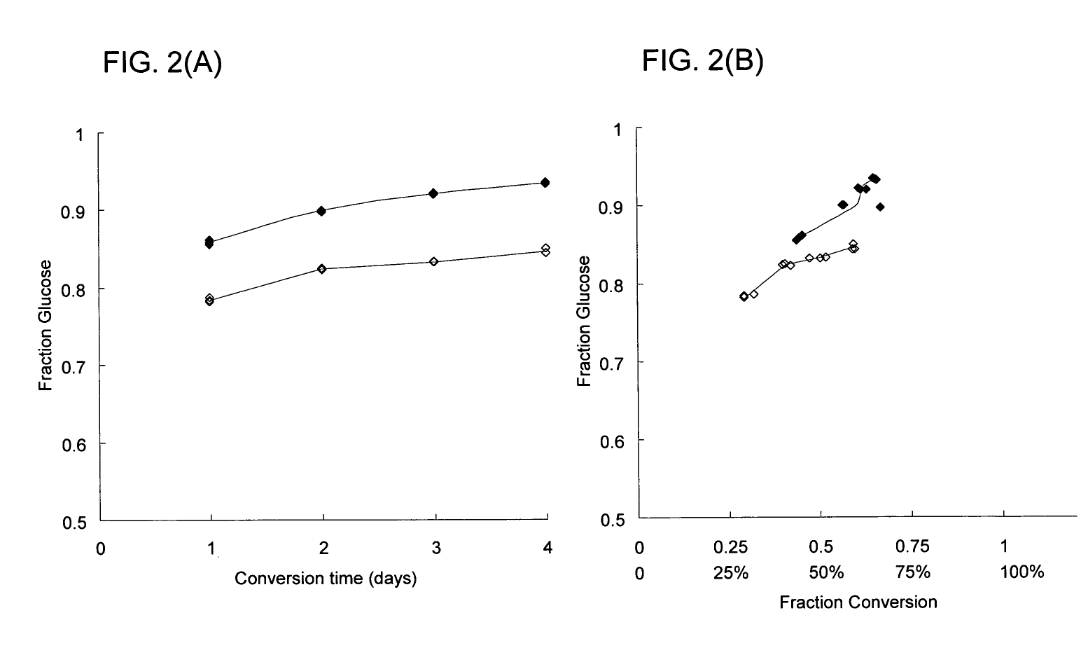

FIGS. 2A-B show the conversion of dilute acid treated corn stover to soluble sugars by 12 mg/g of whole cellulase from Trichoderma reesei over-expressing beta-glucosidase, at 38° C. (open symbols) and 53° C. (closed symbols). T. reesei whole cellulase with elevated β-glucosidase levels converts acid-pretreated corn stover to a higher fraction of glucose at 53° C. than at 38° C.

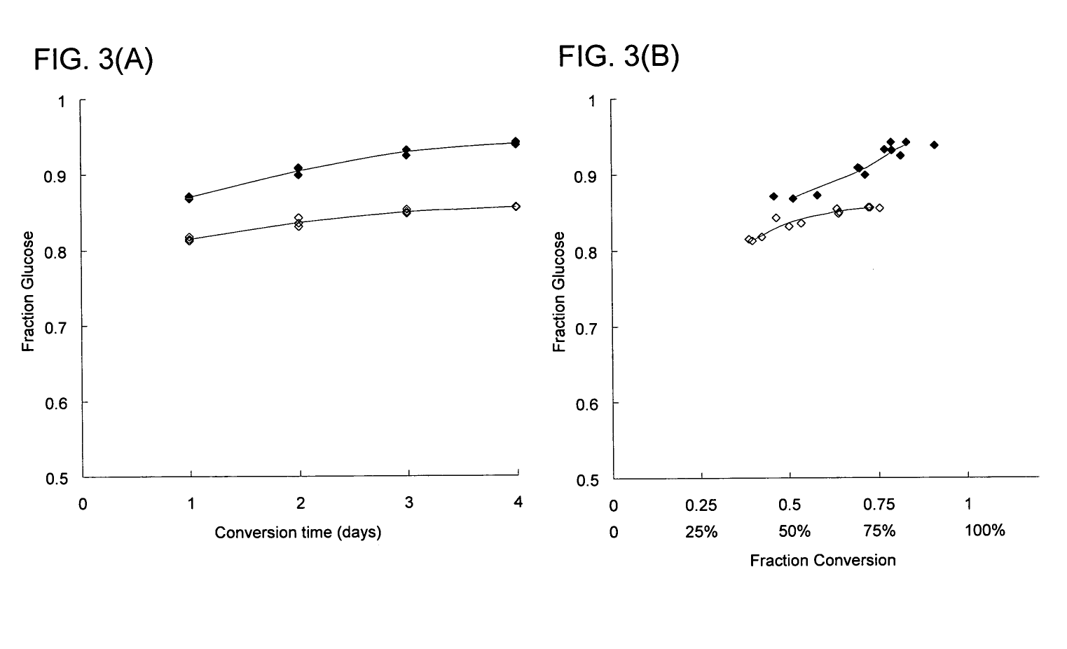

FIGS. 3A-B show the conversion of dilute acid treated corn stover to soluble sugars by 18 mg/g of whole cellulase from Trichoderma reesei over-expressing beta-glucosidase, at 38° C. (open symbols) and 53° C. (closed symbols). T. reesei whole cellulase with elevated β-glucosidase levels converts acid-pretreated corn stover to a higher fraction of glucose at 53° C. than at 38° C.

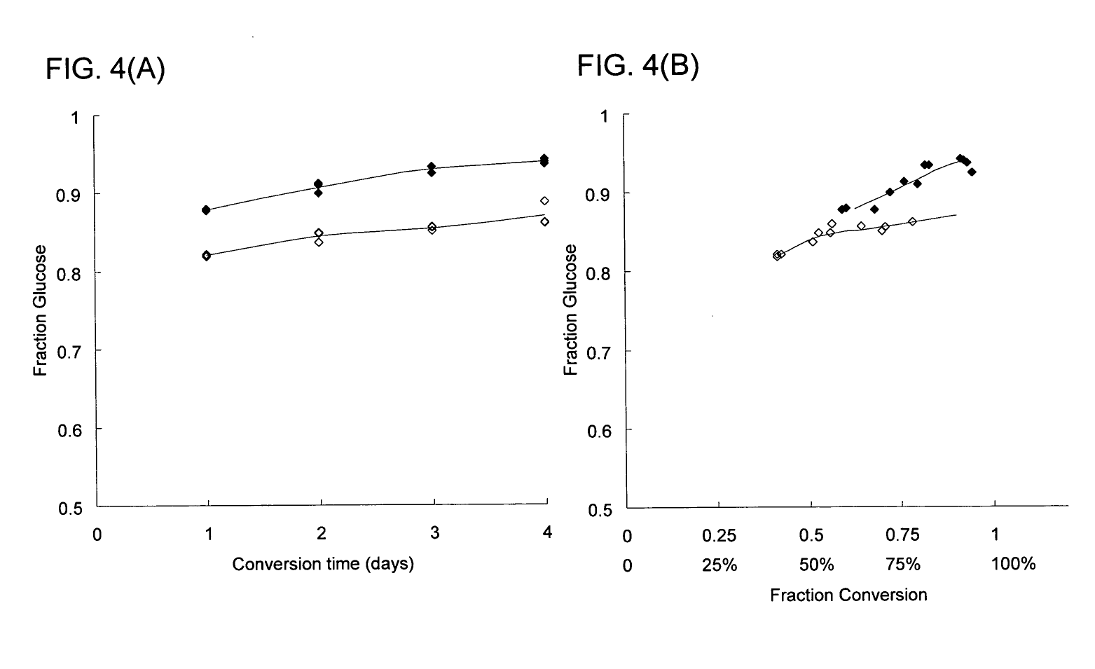

FIGS. 4A-B show the conversion of dilute acid treated corn stover to soluble sugars by mg/g of whole cellulase from Trichoderma reesei over-expressing beta-glucosidase 1 at 38° C. (open symbols) and 53° C. (closed symbols). T. reesei whole cellulase with elevated β-glucosidase levels converts acid-pretreated corn stover to a higher fraction of glucose at 53° C. than at 38° C.

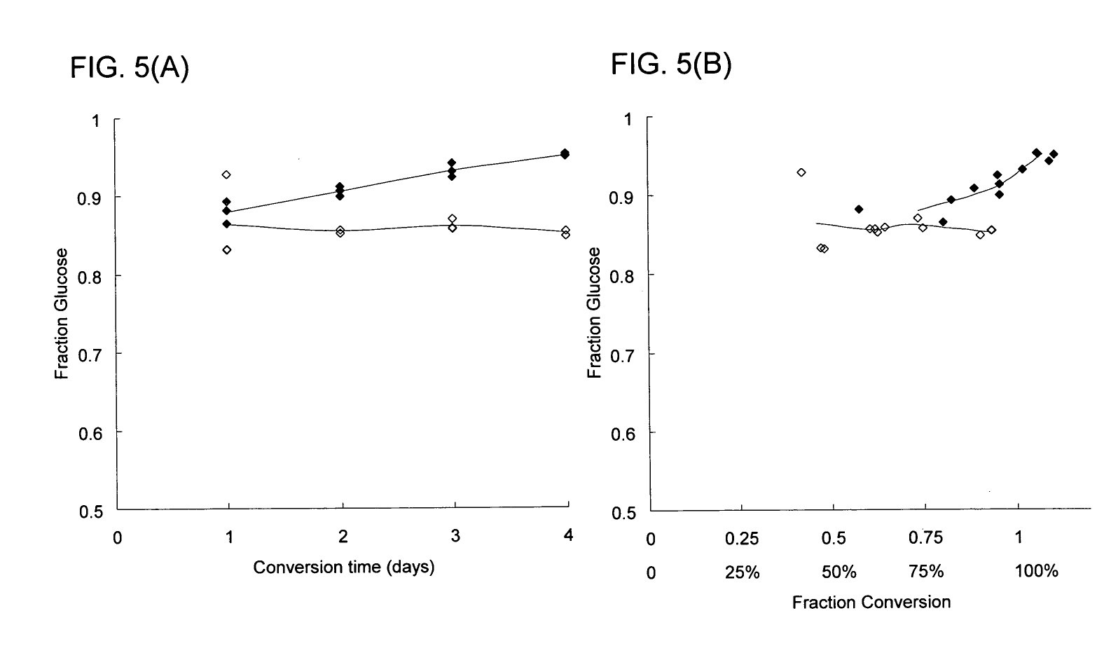

FIGS. 5A-B show the conversion of dilute acid treated corn stover to soluble sugars by mg/g whole cellulase from Trichoderma reesei over-expressing beta-glucosidase, at 38° C. (open symbols) and 53° C. (closed symbols). T. reesei whole cellulase with elevated β-glucosidase levels converts acid-pretreated corn stover to a higher fraction of glucose at 53° C. than at 38° C.

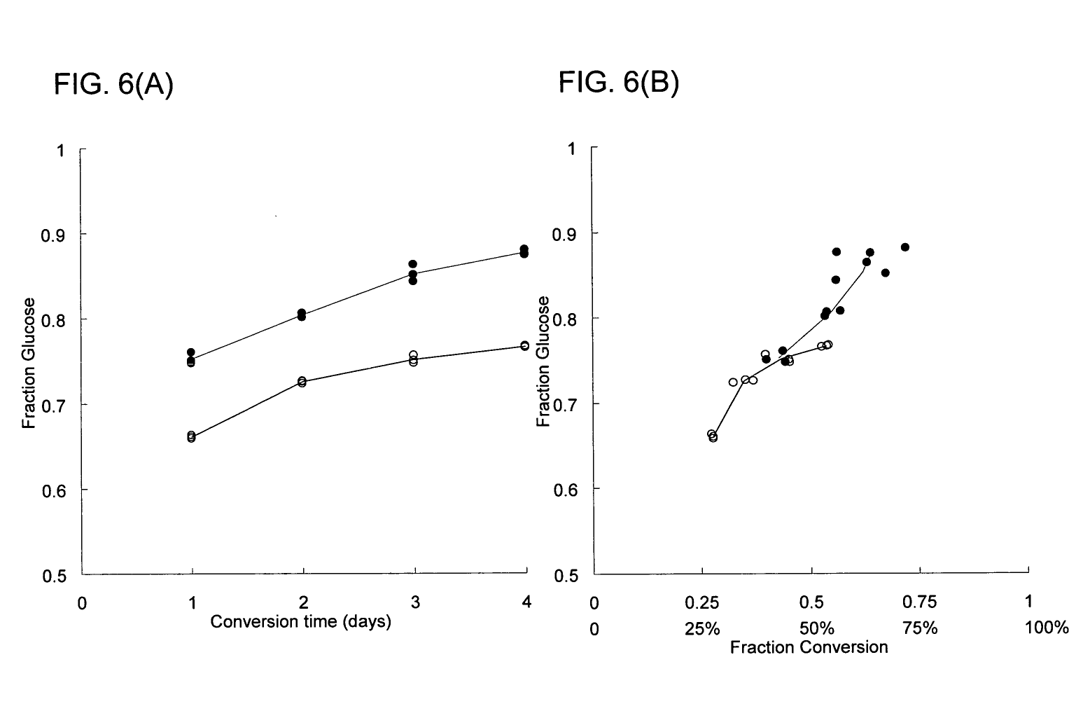

FIGS. 6A-B show the conversion of dilute acid treated corn stover to soluble sugars by 12 mg/g of whole cellulase from Trichoderma reesei, at 38° C. (open symbols) and 53° C. (closed symbols). T. reesei whole cellulase converts acid-pretreated corn stover to a higher fraction of glucose at 53° C. than at 38° C.

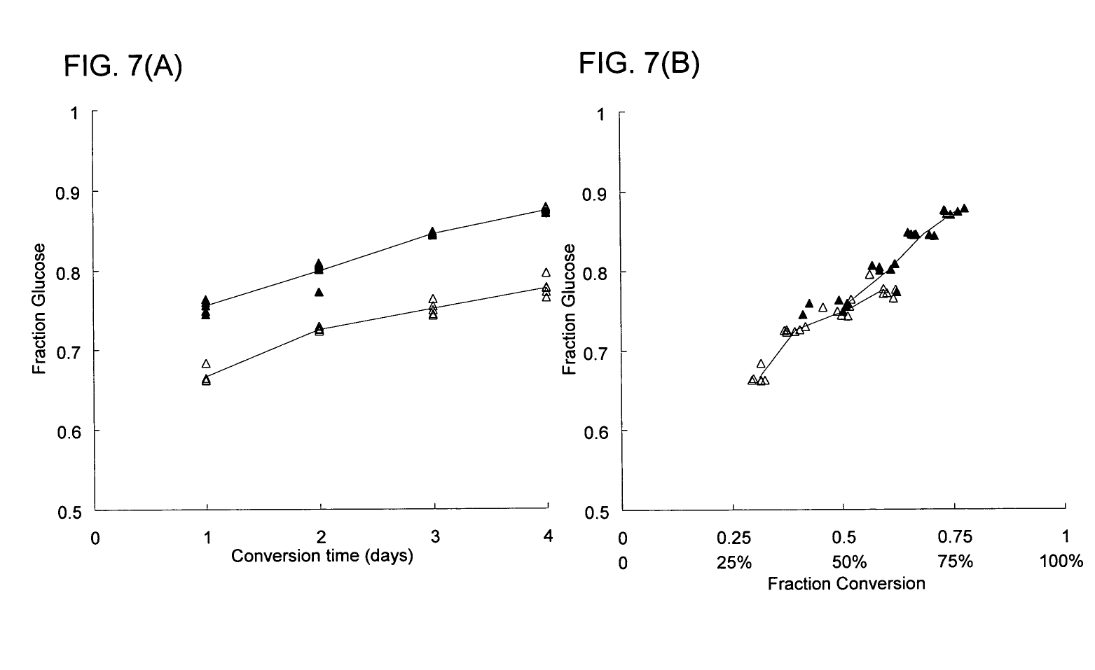

FIGS. 7A-B show the conversion of dilute acid treated corn stover to soluble sugars by 12 mg/g of whole cellulase from Trichoderma reesei expressing a CBH1-E1 fusion protein, at 38° C. (open symbols) and 53° C. (closed symbols). T. reesei whole cellulase converts acid-pretreated corn stover to a higher fraction of glucose at 53° C. than at 38° C.

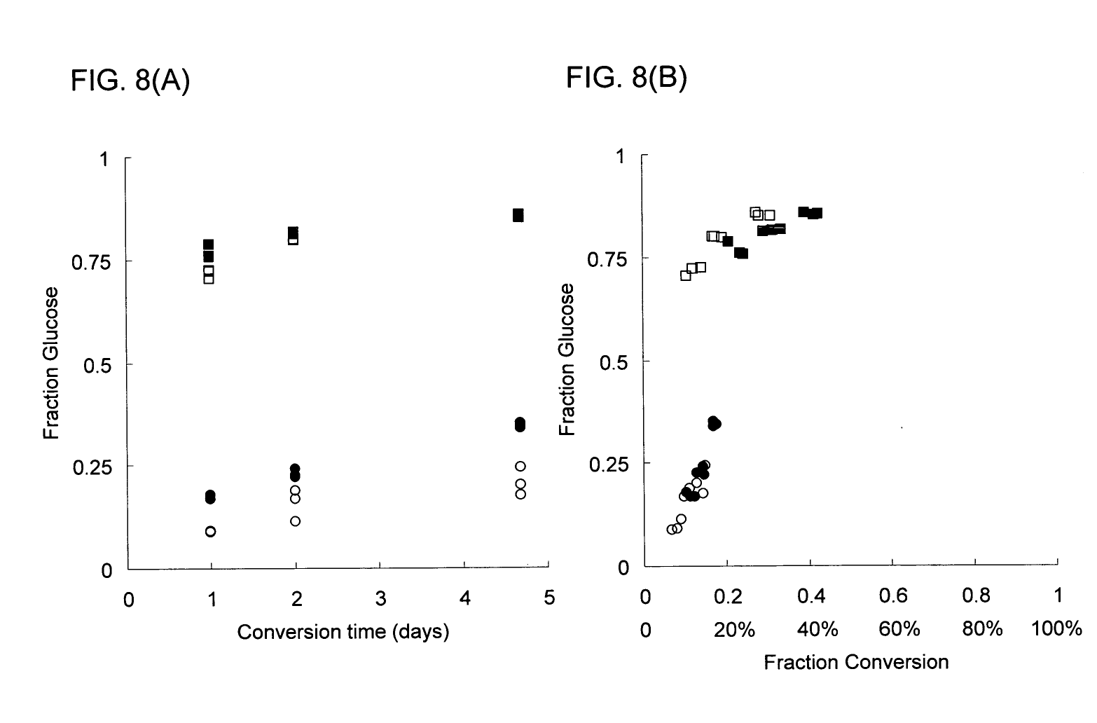

FIGS. 8A-B shows the conversion of dilute acid treated corn stover to soluble sugars by 15 mg/g of a mixture of cellulases composed of either T. reesei EG1 and T. reesei CBH1 (squares) or E1 and H. grisea CBH1 (circles) at 38° C. (open symbols) and 65° C. (closed symbols). Cellulase mixtures containing E1 convert acid-pretreated corn stover to a higher fraction of cellobiose than mixtures containing EG1.

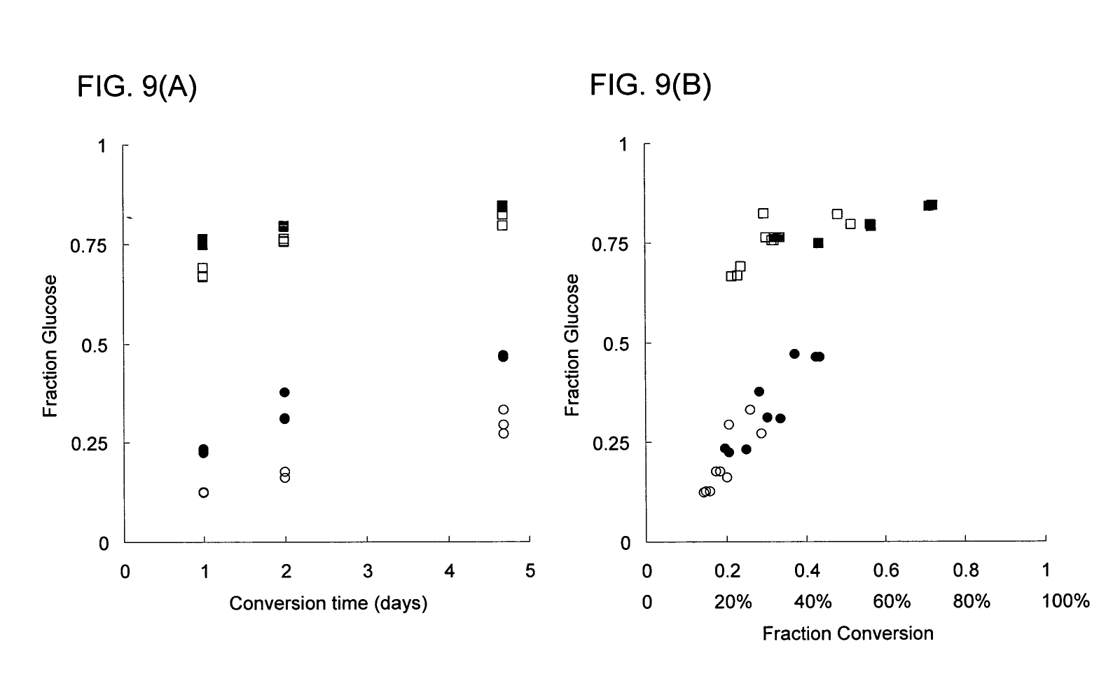

FIGS. 9A-B show the conversion of dilute acid treated corn stover to soluble sugars by 15 mg/g of a mixture of cellulases composed of either EG1, T. reesei CBH1 and T. reesei CBH2 (squares) or E1, H. grisea CBH1 and T. reesei CBH2 (circles) at 38° C. (open symbols) and 65° C. (closed symbols). Cellulase mixtures containing E1 convert acid-pretreated corn stover to a higher fraction of cellobiose than mixtures containing EG1.

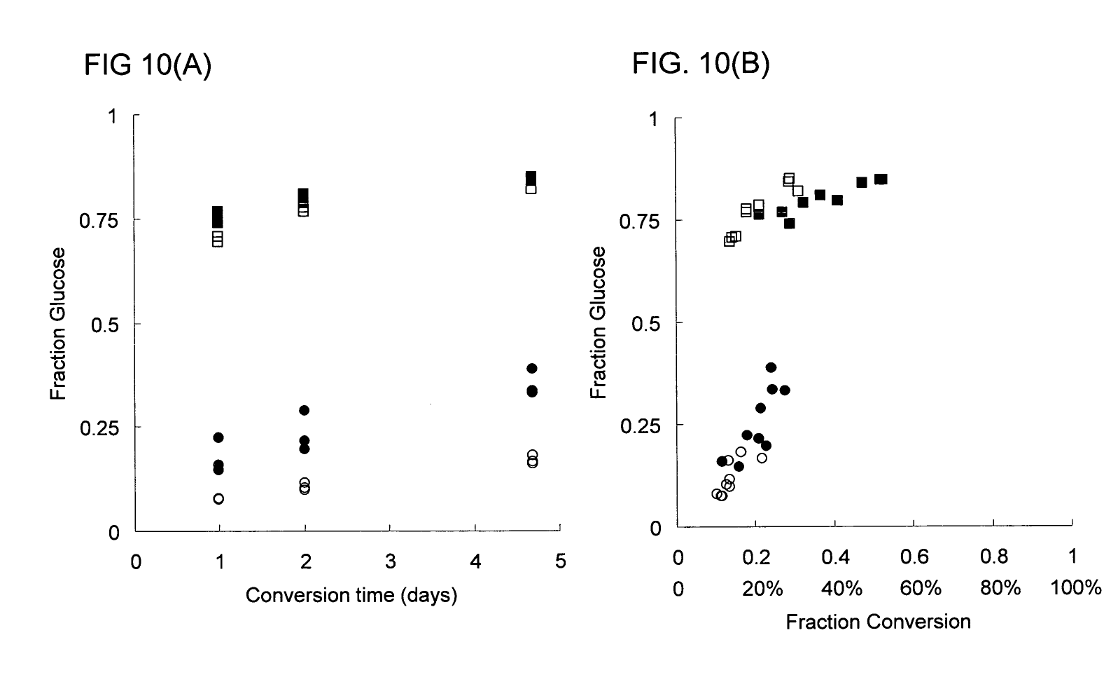

FIGS. 10A-B show the conversion of dilute acid treated corn stover to soluble sugars by 15 mg/g of a mixture of cellulases composed of either EG1, T. reesei CBH11 and T. fusca E3 (squares) or E1, H. grisea CBH1 and T. fusca E3 (circles) at 38° C. (open symbols) and 65° C. (closed symbols). Cellulase mixtures containing E1 convert acid-pretreated corn stover to a higher fraction of cellobiose than mixtures containing EG1.

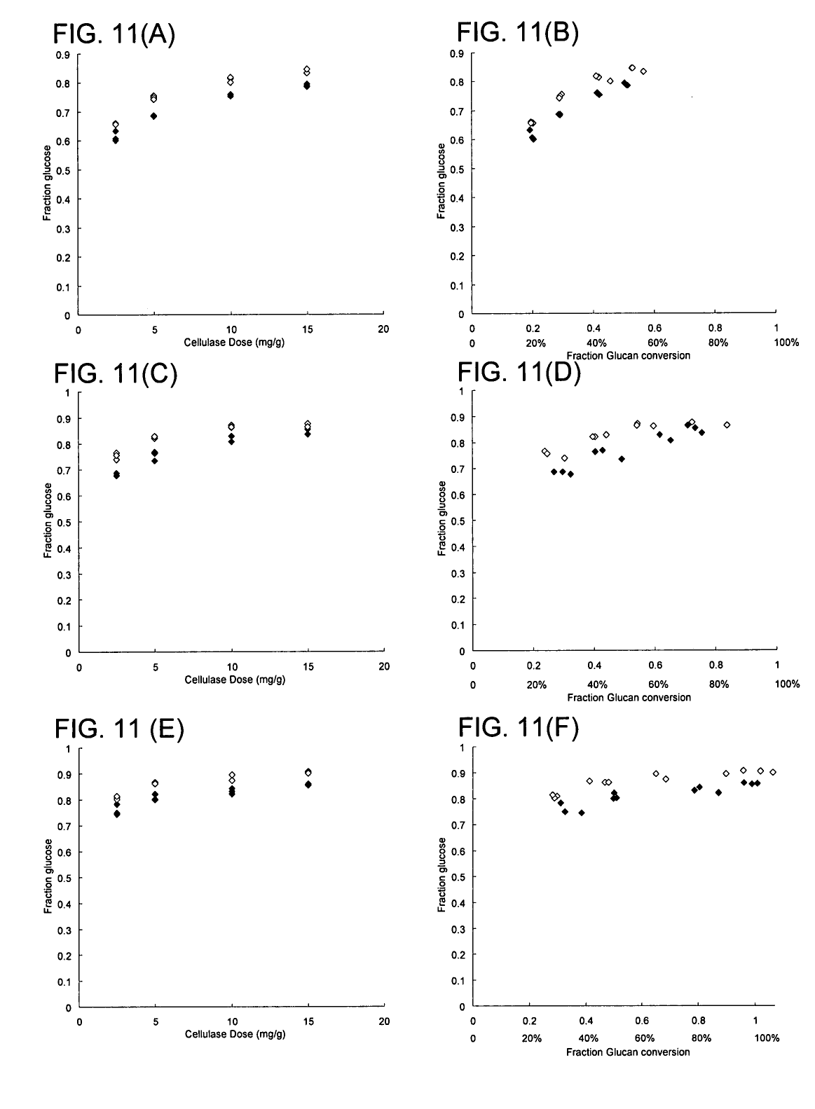

FIGS. 11A-F show the conversion of dilute acid treated corn stover to soluble sugars by Trichoderma reesei whole cellulase at 53° C. (closed symbols) and 59° C. (open symbols) for 1 day (A and B), 2 days (C and D), and 3 days (E and F). The ordinate represents the fraction of glucose with respect to the total sugar (wt/wt basis) (A, B, and E). The abscissa represents the dose of enzyme used (B, D, and E). The abscissa represents the total soluble sugar conversion that is observed (each dose is not explicitly labeled, but a higher dose is indicated by higher conversion). T. reesei whole cellulase converts acid-pretreated corn stover to a higher fraction of glucose at high temperatures.

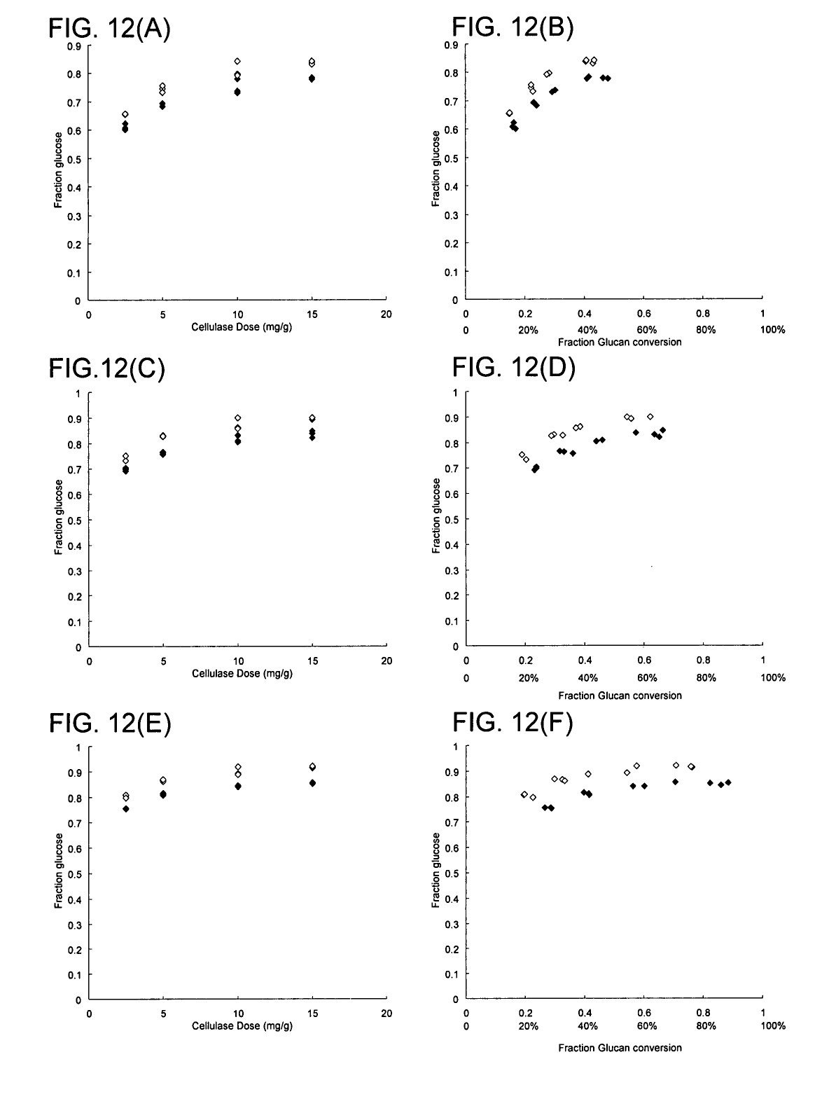

FIGS. 12A-F show the conversion of dilute acid treated corn stover to soluble sugars a Trichoderma reesei whole cellulase expressing a CBH1-E1 fusion protein, at 53° C. (closed symbols) and 59° C. (open symbols) for (A and B) 1, (C and D) 2, and (E and F) 3 days. The ordinate represents the fraction of glucose with respect to the total sugar (wt/wt basis) (A, C, and E). The abscissa represents the dose of enzyme used (B, D, and F) The abscissa represents the total soluble sugar conversion that is observed (each dose is not explicitly labeled, but a higher dose is indicated by higher conversion). T. reesei whole □ ellulose converts acid-pretreated corn stover to a higher fraction of glucose at high temperatures.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for converting a cellulosic material to glucose comprising the steps of:
combining a cellulosic material with a cellulase such that the resulting combination of cellulosic material and cellulase has 1% to about 30% cellulose by weight; and
incubating said cellulosic material and cellulase combination at a temperature greater than about 38° C. to about 100° C. for about 0.1 hours to about 96 hours at a pH of from about 4 to about 9 to cause a hydrolysis reaction to convert at least 20% of said cellulosic material to soluble sugars,
wherein said soluble sugars comprises glucose and cellobiose, and the fraction of glucose is at least 0.75 relative to said soluble sugars.

2. The method of claim 1 wherein the cellulosic material selected from the group consisting of bioenergy crops, agricultural residues, municipal solid waste, industrial solid waste, yard waste, wood, forestry waste, switchgrass, waste paper, sludge from paper manufacture, corn grain, corn cobs, corn husks, corn stover, grasses, wheat, wheat straw, hay, rice straw, sugar cane bagasse, sorghum, soy, trees, switchgrass, hay, barley, barley straw, rice straw, and grasses.

3. The method of claim 1 further comprising pretreating said cellulosic material.

4. The method of claim 3 wherein said pretreatment is selected from a group consisting of steam explosion, pulping, grinding, acid hydrolysis, and combinations thereof.

5. The method of claim 1 further comprising determining the amount of glucose.

6. The method of claim 1 further comprising determining the amount of soluble sugars.

7. The method of claim 1 wherein the amount of cellulase is about 2-40 mg/g cellulosic material.

8. The method of claim 1 wherein said cellulase comprises a whole cellulase.

9. The method of claim 8 wherein said whole cellulase is a Trichoderma reesei whole cellulase.

10. The method of claim 9 wherein said Trichoderma reesei expresses a recombinant enzyme.

11. The method of claim 10 wherein said recombinant enzyme is a beta-glucosidase.

12. A method for converting a cellulosic material to cellobiose comprising the steps of:
combining a cellulosic material with a cellulase mixture comprising an endoglucanase 1 such that the resulting combination of cellulosic material and cellulase mixture has 1% to about 30% cellulose by weight; and
incubating said cellulosic material and cellulase combination at a temperature less than about 100° C. to about 25° C. for about 0.1 hours to about 96 hours at a pH of from about 4 to about 9 to cause a hydrolysis reaction to convert up to 50% of said cellulosic material to soluble sugars, wherein said soluble sugars comprises glucose and cellobiose and the fraction of glucose is less than about 0.5 relative to said soluble sugars.

13. The method of claim 12 wherein the cellulosic material selected from the group consisting of bioenergy crops, agricultural residues, municipal solid waste, industrial solid waste, yard waste, wood, forestry waste, switchgrass, waste paper, sludge from paper manufacture, corn grain, corn cobs, corn husks, corn stover, grasses, wheat, wheat straw, hay, rice straw, sugar cane bagasse, sorghum, soy, trees, switchgrass, hay, barley, barley straw, rice straw, and grasses.

14. The method of claim 12 further comprising pretreating said cellulosic material.

15. The method of claim 14 wherein said pretreatment is selected from a group consisting of steam explosion, pulping, grinding, acid hydrolysis, and combinations thereof.

16. The method of claim 12 further comprising determining the amount of glucose.

17. The method of claim 12 further comprising determining the amount of soluble sugars.

18. The method of claim 12 wherein the amount of said cellulase mixture is about 2 mg-40 mg/g cellulosic material.

19. The method of claim 13 wherein said cellulase mixture further comprises a cellobiohydrolase 1.

20. The method of claim 13 wherein said cellulase mixture further comprises a cellobiohydrolase 2.

21. The method of claim 13 wherein said cellulase mixture further comprises Thermomonospera fusca E3.
